(19) United States
(12) Patent Application Publication
(10) Pub. No.: US 2006/0100181 A1
(43) Pub. Date: May 11, 2006

(54) Combinations for the treatment of inflammatory skin disorders

(76) Inventors: Edward Roydon Jost-Price, West Roxbury, MA (US); Palaniyandi Manivasakam, Brighton, MA (US); Grant R Zimmermann, Somerville, MA (US); Jason Fong, Philadelphia, PA (US); Nicole Hurst, Boston, MA (US); Benjamin A Auspitz, Cambridge, MA (US); M James Nichols, Boston, MA (US); Curtis T Keith, Boston, MA (US)

Correspondence Address: CLARK & ELBING LLP, 101 FEDERAL STREET, BOSTON, MA, 02110, US

(21) Appl. No.: 10/513,237

(22) PCT Filed: May 2, 2003
(86) PCT No.: PCT/US03/13760

§ 371 Date: Aug. 23, 2005

(30) Foreign Application Priority Data
May 3, 2002 (US) 60377475

(51) Int. Cl. A61K 31/573; A61K 31/557

(52) U.S. Cl. 514/171; 514/573

(57) ABSTRACT

The invention features methods and compositions for the treatment of inflammatory skin conditions.

### BACKGROUND OF THE INVENTION

The invention relates to the treatment of inflammatory skin disorders.

Inflammatory skin disorders (e.g., psoriasis, inflammatory dermatoses, and atopic dermatoses) are characterized by dysregulation of the immune system and inappropriate activation of body's defenses, resulting in damage to healthy skin tissue. The damage results in redness, itching, swelling, and blistering of the skin.

Atopic dermatitis occurs in people who have a family history of asthma, allergic rhinitis, or atopic eczema, accompanied by chronic or recurrent dry, extremely itchy, inflamed lesions. The hypersensitivity or allergic reaction that occurs in the skin causes chronic inflammation. Persistent scratching often results in wounds that can become infected with bacteria or viruses. Staphylococcal infections are common in patients with disorders of the skin. Atopic dermatitis affects approximately 10% of children, and in a small percentage of people the symptoms continue into adulthood.

Psoriasis is a common chronic proliferative skin disease, affecting up to 2% of the population. One characteristic of psoriasis is a strong hyperproliferation of epidermal keratinocytes and an incomplete epidermal differentiation that leads to severe scaling of the affected skin areas. This proliferative event is accompanied by an inflammation of the epidermis and dermis, with infiltrates of T-cells, neutrophils, and macrophages. Risk factors that indicate a higher chance of acquiring psoriasis include: stress, infections, certain medications, immunologic factors (e.g., HIV), and family history. There is some association with arthritis.

Treatment of inflammatory skin disorders generally involves reducing contact with irritants (e.g., rough fibers, chemicals, make-up, etc.) and allergens (e.g., moulds, grass pollens, and animal dander), and the application of emollients to keep the skin moisturized. Topical corticosteroids have also been used as anti-inflammatory agents and to treat the symptoms of dermatoses. To date, however, no permanent cure is possible, and there exists a need in the field for new and more effective agents that can be used to treat inflammatory skin disorders.

### SUMMARY OF THE INVENTION

We have discovered that the combination of a steroid with a prostaglandin, a beta-adrenergic receptor ligand, an anti-mitotic agent, or a microtubule inhibitor brings about substantial suppression of TNFα levels induced in white blood cells. TNFα is a major mediator of inflammation. Specific blockade of TNFα using antibodies or soluble receptors is a potent treatment for patients having an inflammatory skin disease, such as psoriasis, inflammatory dermatitis, and atopical dermatitis. Thus, this combination can be used to treat inflammatory skin disorders. Moreover, based on the shared action among prostaglandin family members, beta-adrenergic receptor ligand family members, anti-mitotic agent family members, microtubule inhibitor family members, and steroid family members, any member of a family can be replaced by another member of that family in the combination.

We have also discovered that the combination of a microtubule inhibitor with an azole also provides substantial suppression of TNFα levels induced in white blood cells. Thus, this combination can similarly be used to treat inflammatory skin disorders. Based on the shared action among microtubule inhibitor family members and azole family members, one member of a family can be replaced by another member of that family in the combination.

Accordingly, the invention features a method for treating a patient who is diagnosed with, or is at risk for developing, an inflammatory skin disorder (e.g., psoriasis, inflammatory dermatitis, or atopical dermatitis) by topically administering to the patient a prostaglandin and a steroid, in amounts that treat the patient. In one particular embodiment, the prostaglandin is alprostadil and the steroid is diflorasone, prednisolone, or dexamethasone.

In another aspect, the invention also features another method for treating a patient who is diagnosed with, or is at risk for developing, an inflammatory skin disorder by topically administering to the patient a beta-adrenergic receptor ligand and a steroid, in amounts that treat the patient. One example features isoproterenol and diflorasone, prednisolone, or dexamethasone.

The invention features another method for treating a patient who is diagnosed with, or is at risk for developing, an inflammatory skin disorder. In this method, an anti-mitotic agent and a steroid are administered to the patient in amounts that treat the patient. For example podofilox (podophyllotoxin) can be used in combination with a steroid such as diflorasone, prednisolone, or dexamethasone.

Another method for treating a patient who is diagnosed with, or is at risk for developing, an inflammatory skin disorder includes the step of topically administering to the patient a microtubule inhibitor (e.g., colchicine and vinblastine) and a steroid in amounts that treat the patient. For example colchicine can be used in combination with a steroid such as diflorasone, prednisolone, or dexamethasone.

In yet another aspect, the invention, features a method for treating a patient who is diagnosed with, or is at risk for developing, an inflammatory skin disorder, by topically administering to the patient a microtubule inhibitor (e.g., colchicine and a vinca alkaloid (e.g., vinblastine)) and an azole (e.g., clotrimazole) in amounts that treat the patient. For example vinblastine can be used in combination with clotrimazole.

In each of the foregoing methods, the two drugs can be topically administered separately or together. If administered separately, the compounds can be administered within 14 days of each other (e.g., within 10 days, within five days, twenty-four hours, or one hour of each other). Administration of each compound in the combination can occur 1 to 10 times each day, desirably 1 to 8 times each day, more desirably 1 to 6 times each day, most desirably 1 to 4 times each day, or as necessary to alleviate symptoms.

The invention also features compositions containing one or more of the compounds described above (i.e., a prostaglandin, a beta-adrenergic receptor ligand, an anti-mitotic agent, or a microtubule inhibitor in combination with a steroid, and a microtubule inhibitor in combination with an azole). Particular compositions include alprostidil and diflorasone; isoproterenol and prednisolone; podofilox and dexamethasone; colchicine and flumethasone; vinblastine and clotrimazole. Desirably, these compositions are formulated for topical administration and are in effective amounts for the treatment of an inflammatory skin disorder.

The invention also features a method of producing pharmaceutical compositions for treating an inflammatory skin disorder containing one or more of the compounds described above (i.e., a prostaglandin, a beta-adrenergic receptor ligand, an anti-mitotic agent, or a microtubule inhibitor in combination with a steroid, and a microtubule inhibitor in combination with an azole). The method is used to produce particular compositions including alprostidil and diflorasone; isoproterenol and prednisolone; podofilox and dexamethasone; colchicine and flumethasone; vinblastine and clotrimazole. Desirably, the method is used to produce compositions formulated for topical administration and which incorporate the compounds in effective amounts for the treatment of an inflammatory skin disorder.

The specific amounts of the prostaglandin, the beta-adrenergic receptor ligand, the anti-mitotic agent, the microtubule inhibitor, the steroid, and the azole administered depend on the specific combination of components and can be determined by one skilled in the art. Generally, when delivered by topical application, the prostaglandin, beta-adrenergic receptor ligand, anti-mitotic agent, and microtubule inhibitor, are administered at a dose of 1 pg to 100 mg per day, desirably 1 pg to 75 mg per day, more desirably 1 pg to 50 mg per day, and most desirably 1 pg to 10 mg per day. The steroid is topically administered at a total daily dosage of about 0.1 mg to 1500 mg per day, desirably about 0.1 mg to 200 mg per day, more desirably about 0.1 mg to 100 mg per day, and most desirably 0.1 mg to 30 mg per day. Dosages of up to 3000 mg per day may be necessary. The azole is topically administered at a dosage of about 0.01 mg to 2000 mg per day, desirably about 0.01 mg to 800 mg per day, more desirably about 0.01 mg to 200 mg per day, and most desirably about 0.01 mg to 50 mg per day.

In several desired dose combinations, the ratio of prostaglandin (e.g., alprostadil) to steroid (e.g., diflorasone) is desirably 10:1 to 20:1 by weight; the ratio of beta-adrenergic receptor ligand (e.g., isoproterenol) to steroid (e.g., prednisolone) is desirably 10:1 to 100:1 by weight; the ratio of anti-mitotic agent (e.g., podofilox) to steroid (e.g., dexamethasone) is desirably 10:1 to 500:1 by weight; the ratio of microtubule inhibitor (e.g., colchicine) to steroid (e.g., flumethasone) is desirably 50:1 to 1000:1 by weight; the ratio of microtubule inhibitor (e.g., vinblastine) to azole (e.g., clotrimazole) is desirably 2:1 to 1:2 by weight.

Compounds useful in the invention include those described herein in any of their pharmaceutically acceptable forms, including isomers such as diastereomers and enantiomers, salts, solvates, and polymorphs thereof, as well as racemic mixtures of the compounds described herein.

By an “amount sufficient to treat” is meant the amount of a compound, in a combination of the invention, required to reduce or prevent the symptoms of an inflammatory skin disorder. A sufficient amount of active compound(s) used to practice the present invention for therapeutic treatment of conditions caused by or contributed to by an inflammatory skin disorder varies depending upon the manner of administration, the age, body weight, and general health of the patient. Ultimately, the attending physician or veterinarian will decide the appropriate amount and dosage regimen. Such amount is referred to as a sufficient amount.

By “anti-mitotic agent” is meant an agent that is capable of inhibiting mitosis. Exemplary anti-mitotic agents include, for example, podofilox, etoposide, teniposide, and griseofulvin.

By “azole” is meant any member of the class of anti-fungal compounds having a five-membered ring of three carbon atoms and two nitrogen atoms (e.g., the imidazoles) or two carbon atoms and three nitrogen atoms (e.g., triazoles), which are capable of inhibiting fungal growth. A compound is considered “antifungal” if it inhibits growth of a species of fungus in vitro by at least 25%. Typically, azoles are administered in dosages of greater than 200 mg per day when used as an antifungal agent. The azole can be selected from an imidazole or a triazole. Examples of exemplary imidazoles are sulconazole, miconazole, clotrimazole, oxiconazole, butocontazole, tioconazole, econazole, and ketoconazole. Examples of exemplary triazoles are itraconazole, fluconazole, voriconazole, posaconazole, ravuconazole, and terconazole.

By “beta-adrenergic receptor ligand” is meant an agent that binds the beta-adrenergic receptor in a sequence-specific manner. Exemplary beta-adrenergic receptor ligands include agonists and antagonists. Exemplary beta-adrenergic receptor agonists include, for example, isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, and ephedrine. Exemplary beta-adrenergic receptor antagonists include, for example, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, and propafenone.

The term “inflammatory skin disorder” encompasses a variety of conditions, including autoimmune diseases and proliferative skin diseases. Inflammatory skin disorders result in the damage of healthy skin tissue by an inflammatory process. Examples of inflammatory skin disorders include scleroderma, systemic lupus erythematosus, and inflammatory dermatoses. Inflammatory dermatoses include, for example, psoriasis, atopic dermatitis, non-specific dermatitis, lamellar ichthyosis, epidemlolytic hyperkeratosis, premalignant keratosis, acne, and seborrheic dermatitis, pityriasis roseas, acute febrile neutrophilic dermatosis, eczema (e.g., asteatotic eczema, dyshidrotic eczema, vesicular palmoplantar eczema), balanitis circumscripta plasmacellularis, balanoposthitis, Behcet disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, juvenile palmar-plantar dermatosis, keratosis pilaris, acne fulminans, acrodermatitis enteropathica, infantile acropustulosis, mastocytomas, diffuse cutaneous mastocytosis, erythema multiforme mino, erythema multiforme major, bullous dermatosis, alopecia, vitiligo, and transient acantholytic dermatosis.

By “reduce or prevent the symptoms of an inflammatory skin disorder” is meant to lessen or inhibit pain, inflammation, itching, redness, swelling, blistering, dry skin, scaling, and lesions caused by or associated with an inflammatory skin disorder.

By “microtubule inhibitor” is meant an agent that is capable of affecting the equilibrium between free tubulin dimers and assembled polymers. Exemplary microtubule inhibitors include, for example, colchicine, vinca alkaloids (e.g., vinblastine, vincristine, vinorelbine, and vindesine), paclitaxel, and docetaxel.

By “prostaglandin” is meant a member of the lipid class of biochemicals that belongs to a subclass of lipids known as the eicosanoids, because of their structural similarities to the C-20 polyunsaturated fatty acids, the eicosaenoic acids. Exemplary prostaglandins include alprostidil, dinoprostone, misoprostil, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, ornoprostil, 13,14-dihydroprostaglandin F2α, and prostaglandin J.

By “steroid” is meant any naturally occurring or synthetic hormone that can be derived from cholesterol and is characterized by a hydrogenated cyclopentanoperhydrophenanthrene ring system. Naturally occurring steroids are generally produced by the adrenal cortex. Synthetic steriods may be halogenated. Steroids may have corticoid, glucocorticoid, and/or mineralocorticoid activity. Examples of steroids are algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocoitolone, clocortol one pival ate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydiocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Desirably, the corticosteroid is selected from cortisone, dexamethasone, hydrocortisone, methylprednisolone, prednisone, traimcinolone, and diflorasone.

By “treating” is meant administering a pharmaceutical composition such that the symptoms of an inflammatory skin disorder are reduced or prevented. Administration may be to a patient already suffering from an inflammatory skin disorder to improve the patient's condition (i.e., relieve pain, inflammation, itching, redness, swelling, blistering, dry skin, scaling, and lesions caused by ail inflammatory skin disorder, and help to maintain a patient's normal lifestyle) or to prevent the occurrence or reoccurrence of an inflammatory skin disorder in a patient. By “patient” is meant any animal (e.g., a human).

The combinations described above for the treatment of an inflammatory skin disorder allows for the administration of a low dose of each compound and less total active compound, thus providing similar efficacy with less toxicity, and reduced costs.

Other features and advantages of the invention will be apparent from the following detailed description, and from the claims.

### DETAILED DESCRIPTION OF THE INVENTION

We have discovered that the combination of a prostaglandin, a beta-adrenergic receptor ligand, an anti-mitotic agent, or a microtubule inhibitor with a steroid, as well as the combination of a microtubule inhibitor with an azole, has substantial TNFα suppressing activity on white blood cells. Concentrations that effectively suppress TNFα activity are not unacceptably toxic to normal cells. Thus, combinations of prostaglandins, beta-adrenergic receptor ligands, anti-mitotic agents, or microtubule inhibitors with steroids, as well as combinations of microtubule inhibitors with azoles are useful for the treatment of inflammatory skin disorders.

Therapy

Combination therapy according to the invention may be performed alone or in conjunction with another therapy and may be provided at home, the doctor's office, a clinic, a hospital's outpatient department, or a hospital. Treatment generally begins at a hospital so that the doctor can observe the therapy's effects closely and make any adjustments that are needed. The duration of the combination therapy depends on the type of disease or disorder being treated, the age and condition of the patient, the stage and type of the patient's disease, and how the patient responds to the treatment. Additionally, a person having a greater risk of developing an inflammatory skin disorder (e.g., a person who is genetically predisposed or having a prior diagnosis of an inflammatory skin disorder) may receive prophylactic treatment to inhibit or delay the onset of symptoms.

An inflammatory skin disease is alleviated when there is a noticeable decrease in a lesion of the skin, or a decrease in the presence of itching, redness, swelling, blistering, or other manifestation of the disease or condition. The alleviation of symptoms can occur without a decrease in residual redness, dilated blood vessels, hyper-pigmentation, or hypo-pigmentation. For the purposes of this invention, psoriasis is considered alleviated when a scale-free psoriasis lesion is noticeably decreased in thickness.

The dosage, frequency, and mode of administration (e.g., gel, spray, or cream) of each component of the combination can be controlled independently. For example, one compound may be administered topically by gel three times per day, while the second compound may be administered topically by spray once per day. Combination therapy may be given in on-and-off cycles that include rest periods so that the patient's body has a chance to recovery from any as yet unforeseen side-effects. The compounds may also be formulated together such that one administration delivers both compounds.

Formulation of Pharmaceutical Compositions

The compositions of the present invention are formulated for topical administration. Suitable formulations include gels, sprays, ointments, and creams. Administration of each compound of the combination may be by any suitable means that results in a concentration of the compound that, combined with the other compound, is effective. Each compound can be admixed with a suitable carrier substance, and is generally present in an amount of 1-95% by weight of the total weight of the composition. If desirable, the compounds can be formulated together.

The pharmaceutical compositions may be formulated for topical use according to conventional pharmaceutical practice (see, e.g., Remington: The Science and Practice of Pharmacy, (20th ed.) ed. A. R. Gennaro, 2000, Lippencott Williams & Wilkens, Philadelphia, Pa., and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time period after administration, using controlled release topical formulations.

Therapeutic compositions suitable for topical application include conventional anhydrous or aqueous preparations including ointments, lotions, creams, pastes, jellies, sprays, aerosols, and oils. There preparations can include oleaginous, aqueous, or emulsion-type bases. Optionally, topically applied formulations can be covered with an occlusive or semi-occlusive dressing.

Dosages

We have discovered that combinations of compounds can be used to effectively treat inflammatory skin disorders. These combinations include alprostadil and diflorasone; isoproterenol and prednisolone; podofilox and dexamethasone; colchicine and flumethasone; and vinblastine and clotrimazole. As is described herein, each of these compounds is a member of a larger family. Based on the shared action among family members, any member of a family can be replaced by another member of that family in the combination.

The dosage of each compound used in any given therapeutic method depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

As is described above, the compound(s) are administered topically. Generally, when delivered by topical application, the prostaglandin, beta-adrenergic receptor ligand, anti-mitotic agent, and microtubule inhibitor are administered at a dose of 1 pg to 100 mg per day, desirably 1 pg to 75 mg per day, more desirably 1 pg to 50 mg per day, and most desirably 1 pg mg to 10 mg per day (for a concentration range of between 0 and 500 μM, desirably 1 to 100 μM, more desirably 1 to 50 μM, and most desirably 1 to 20 μM). The steroid is topically administered at a total daily dosage of about 0.1 mg to 1500 mg per day, desirably about 0.1 mg to 200 mg per day, more desirably about 0.1 mg to 100 mg per day, and most desirably 0.1 mg to 30 mg per day. Dosages of up to 3000 mg per day may be necessary. The azole is topically administered at a dosage of about 0.01 mg to 2000 mg per day, desirably about 0.01 mg to 800 mg per day, more desirably about 0.01 mg to 200 mg per day, and most desirably about 0.01 mg to 50 mg per day.

Combination effects are seen at all ratios tested, however, the best effects are seen with particular ratios. Desired ratios for the combinations are as follows: the ratio of prostaglandin (e.g., alprostadil) to steroid (e.g., diflorasone) is desirably 10:1 to 20:1 by weight; the ratio of beta-adrenergic receptor ligand (e.g., isoproterenol) to steroid (e.g., prednisolone) is desirably 10:1 to 100:1 by weight; the ratio of anti-mitotic agent (e.g., podofilox) to steroid (e.g., dexamethasone) is desirably 10:1 to 500:1 by weight; the ratio of microtubule inhibitor (e.g., colchicine) to steroid (e.g., flumethasone) is desirably 50:1 to 1000:1 by weight; and the ratio of microtubule inhibitor (e.g., vinblastine) to azole (e.g., clotrimazole) is desirably 2:1 to 1:2 by weight.

Administration can be one to four times daily for one day to one year, and may even be for the life of the patient. Chronic, long-term administration will be indicated in many cases.

The following examples are to illustrate the invention. They are not meant to limit the invention in any way.

### EXAMPLE 1

### Assay for TNFα Suppressing Activity

The compound dilution matrix was assayed using a TNFα ELISA method. Briefly, a 100 μl suspension of diluted human white blood cells contained within each well of a polystyrene 384-well plate (NalgeNunc) was stimulated to secrete TNFα by treatment with a final concentration of 10 ng/nl phorbol 12-myristate 13-acetate (Sigma) and 750 ng/ml ionomycin (Sigma). Various concentrations of each test compound were added at the time of stimulation. After 16-18 hours of incubation at 37° C. in a humidified incubator, the plate was centrifuged and the supernatant transferred to a white opaque polystyrene 384 well plate (NalgeNunc, Maxisorb) coated with an anti-TNFα antibody (PharMingen, #18631D). After a two-hour incubation, the plate was washed (Tecan PowerWasher 384) with phosphate buffered saline (PBS) containing 0.1% Tween 20 (polyoxyethylene sorbitan monolaurate) and incubated for an additional one hour with another anti-TNFα antibody that was biotin labeled (PharMingen, 18642D) and horseradish peroxidase (HRP) coupled to strepavidin (PharMingen, #13047E).). After the plate was washed with 0.1% Tween 20/PBS, an HRP-luminescent substrate was added to each well and light intensity measured using a LJL Analyst plate luminometer. Sets of control wells contained a serial dilution of Cyclosporin A (Sigma) starting at a final concentration of 0.5 μg/ml.

### EXAMPLE 2

### Preparation of Combinations of Compounds

Stock solutions containing a prostaglandin, a beta-adrenergic receptor ligand, an anti-mitotic agent, a microtubule inhibitor, a steroid, or an azole were made in dimethylsulfoxide (DMSO) at a final concentration of between 0 and 20 μM. Master plates were prepared to contain dilutions of the stock solutions of the compounds described above. Master plates were sealed and stored at −20° C. until ready for use.

The final pairwise combination plates were generated by transferring stock solution from the specific master plates to a dilution plate containing 100 μl of media (RPMI; Gibco BRL, #11875-085), 10% Fetal Bovine Serum (Gibco BRL, #25140-097), 2% Penicillin/Streptomycin (Gibco BRL, #15140-122)) using the TomTec Quadra Plus liquid handler. This dilution plate was then mixed and a 10 μl aliquot transferred to the final assay plate, which had been pre-filled with 40 μl/well RPMI media containing the appropriate stimulant to activate TNFα secretion (see below).

### EXAMPLE 3

### Testing of Combinations for TNFα Suppressing Activity

Pair combinations were tested for the ability to suppress TNF secretion from stimulated white blood cells. TNF suppressing activity was investigated using low doses of alprostadil with diflorasone (see Table 1); isoproterenol with prednisolone (see Table 2), podofilox with dexamethasone (see Table 3), colchicine with flumethasone (see Table 4), and vinblastine with clotrimazole (see Table 5) significantly increased the suppression of TNF secretion from stimulated white blood cells.

TABLE 1
Diflorasone (μM)

| | | 0.12 | 0.06 | 0.03 | 0.015 | 0.0075 | 0.0037 | 0.0018 | 0.0009 | 0.0004 | 0 |
|---|---|---|---|---|---|---|---|---|---|---|---|
| Alprostadil | 1 | 72.62 | 72.67 | 70.77 | 70.29 | 67.23 | 63.22 | 59.11 | 53.39 | 52.08 | 37.49 |
| (μM) | 0.5 | 68.73 | 67.05 | 68.08 | 69.60 | 66.32 | 60.6 | 56.41 | 51.82 | 45.67 | 34.57 |
| | 0.25 | 69.15 | 68.18 | 68.83 | 67.08 | 62.40 | 59.85 | 52.13 | 50.28 | 44.76 | 33.03 |
| | 0.13 | 65.79 | 66.39 | 65.69 | 65.59 | 61.22 | 55.74 | 49.80 | 45.55 | 46.09 | 36.80 |
| | 0.06 | 65.39 | 63.34 | 63.46 | 62.61 | 56.36 | 50.04 | 48.63 | 47.49 | 39.66 | 34.11 |
| | 0.03 | 61.84 | 62.71 | 62.43 | 60.49 | 56.76 | 50.03 | 47.30 | 40.78 | 37.18 | 30.66 |
| | 0.02 | 60.27 | 61.21 | 59.78 | 57.56 | 54.35 | 49.38 | 44.12 | 41.64 | 38.20 | 26.54 |
| | 0.01 | 59.99 | 57.18 | 55.74 | 53.33 | 48.4 | 45.81 | 37.46 | 35.22 | 29.74 | 19.25 |
| | 0.005 | 55.36 | 52.22 | 56.57 | 53.13 | 46.53 | 38.45 | 35.02 | 33.69 | 29.39 | 14.53 |
| | 0 | 42.79 | 40.28 | 42.11 | 35.75 | 32.96 | 28.52 | 15.85 | 12.86 | 5.28 | −2.83 |

TABLE 2
Prednisolone (μM)

| | | 0.1 | 0.05 | 0.03 | 0.015 | 0.075 | 0.038 | 0.019 | 0.009 | 0.004 | 0 |
|---|---|---|---|---|---|---|---|---|---|---|---|
| Isoproteronol | 2 | 56.55 | 49.98 | 40.08 | 34.84 | 36.5 | 29.63 | 28.04 | 22.05 | 22.79 | 18.92 |
| (μM) | 1 | 51.69 | 47.29 | 42.75 | 39.21 | 34.56 | 34.27 | 32.59 | 26.86 | 26.99 | 23.42 |
| | 0.5 | 49.63 | 39.36 | 41.34 | 30.87 | 30.26 | 24.39 | 25.72 | 20.39 | 12.93 | 13.88 |
| | 0.25 | 48.74 | 46.84 | 34.12 | 27.91 | 28.02 | 23.50 | 19.96 | 19.90 | 17.09 | 8.58 |
| | 0.13 | 48.99 | 38.70 | 29.84 | 28.60 | 17.99 | 24.11 | 13.79 | 21.75 | 13.88 | 6.27 |
| | 0.06 | 48.36 | 35.48 | 31.05 | 23.60 | 21.35 | 20.60 | 16.22 | 14.38 | 11.04 | 10.52 |
| | 0.03 | 41.27 | 30.51 | 31.58 | 18.18 | 22.08 | 23.46 | 16.56 | 21.76 | 15.26 | 10.44 |
| | 0.02 | 44.75 | 35.06 | 30.03 | 24.33 | 21.31 | 19.11 | 16.18 | 17.35 | 9.50 | 7.24 |
| | 0.01 | 40.89 | 29.10 | 26.05 | 24.43 | 20.71 | 14.04 | 14.35 | 10.37 | 7.26 | 5.35 |
| | 0 | 40.87 | 32.38 | 22.89 | 21.29 | 16.24 | 18.71 | 19.09 | 12.20 | 6.05 | 0 |

TABLE 3
Dexamethasone (μM)

| | | 0.1 | 0.05 | 0.03 | 0.015 | 0.0075 | 0.0038 | 0.0019 | 0.0009 | 0.0005 | 0 |
|---|---|---|---|---|---|---|---|---|---|---|---|
| Podofilox | 2.41 | 66.35 | 65.60 | 58.89 | 58.67 | 52.35 | 49.36 | 43.63 | 43.38 | 41.73 | 34.00 |
| (μM) | 1.21 | 68.40 | 64.47 | 60.49 | 55.40 | 53.92 | 49.33 | 45.42 | 42.70 | 42.58 | 32.73 |
| | 0.6 | 66.95 | 65.55 | 61.60 | 58.54 | 51.04 | 48.45 | 44.84 | 45.08 | 43.96 | 38.66 |
| | 0.3 | 66.27 | 63.17 | 62.02 | 55.89 | 53.76 | 49.49 | 47.58 | 44.23 | 42.23 | 39.48 |
| | 0.15 | 65.27 | 62.18 | 58.20 | 52.39 | 55.23 | 50.34 | 48.43 | 44.68 | 43.07 | 41.15 |
| | 0.08 | 61.43 | 56.80 | 59.20 | 50.44 | 49.69 | 43.20 | 44.08 | 44.02 | 36.17 | 29.63 |
| | 0.04 | 42.29 | 39.13 | 45.60 | 29.40 | 30.17 | 36.57 | 15.82 | 14.21 | 7.54 | 5.91 |
| | 0.02 | 43.42 | 39.18 | 33.28 | 24.92 | 19.40 | 32.89 | 7.47 | 5.22 | 5.82 | −3.32 |
| | 0.01 | 42.18 | 38.83 | 31.75 | 29.10 | 18.61 | 10.76 | 8.01 | 4.78 | −2.77 | 0.02 |
| | 0 | 44.50 | 35.42 | 32.43 | 27.36 | 22.18 | 7.26 | 11.32 | 6.4 | 4.97 | −1.21 |

TABLE 4
Flumethasone (μM)

| | | 0.04 | 0.02 | 0.01 | 0.0050 | 0.0025 | 0.0013 | 0.0006 | 0.0003 | 0.0002 | 0 |
|---|---|---|---|---|---|---|---|---|---|---|---|
| Colchicine | 2.5 | 72.68 | 70.14 | 68.32 | 63.85 | 59.22 | 52.28 | 49.58 | 47.18 | 45.96 | 41.21 |
| (μM) | 1.25 | 69.35 | 66.52 | 60.86 | 57.04 | 53.25 | 45.46 | 43.77 | 41.35 | 39.18 | 34.11 |
| | 0.63 | 63.94 | 60.64 | 56.34 | 53.06 | 46.59 | 40.73 | 36.21 | 34.05 | 31.39 | 28.69 |
| | 0.31 | 57.81 | 54.31 | 52.24 | 45.42 | 39.92 | 32.60 | 31.66 | 28.96 | 23.78 | 23.49 |
| | 0.16 | 56.29 | 51.01 | 48.73 | 41.14 | 36.60 | 31.01 | 28.89 | 17.43 | 19.00 | 14.33 |
| | 0.08 | 50.62 | 46.79 | 40.22 | 31.05 | 25.80 | 22.33 | 20.98 | 12.76 | 9.38 | 7.94 |
| | 0.04 | 53.98 | 48.14 | 42.82 | 38.49 | 31.77 | 26.54 | 21.04 | 17.00 | 10.56 | 6.56 |
| | 0.02 | 47.77 | 46.68 | 42.12 | 38.69 | 27.81 | 21.19 | 11.01 | 11.11 | 4.58 | 4.23 |
| | 0.01 | 45.53 | 45.71 | 36.8 | 26.64 | 24.14 | 14.34 | 10.60 | 6.02 | 1.87 | 0.75 |
| | 0 | 49.24 | 41.29 | 36.52 | 29.56 | 21.52 | 11.17 | 12.52 | 5.10 | −1.75 | −5.53 |

TABLE 5
Clotrimazole (μM)

| | | 2 | 1 | 0.5 | 0.25 | 0.13 | 0.06 | 0.03 | 0.02 | 0.01 | 0 |
|---|---|---|---|---|---|---|---|---|---|---|---|
| Vinbalstine | 1.1 | 80.19 | 77.75 | 72.74 | 68.28 | 61.34 | 59.49 | 53.71 | 59.72 | 57.70 | 48.98 |
| (μM) | 0.55 | 79.63 | 74.72 | 70.32 | 64.4 | 61.23 | 60.63 | 55.98 | 54.76 | 50.43 | 51.76 |
| | 0.28 | 77.35 | 68.40 | 66.67 | 59.76 | 56.88 | 54.22 | 55.26 | 35.45 | 38.94 | 44.01 |
| | 0.14 | 74.28 | 68.48 | 60.16 | 60.50 | 55.49 | 50.27 | 49.76 | 47.45 | 49.19 | 45.63 |
| | 0.07 | 66.63 | 62.30 | 57.65 | 47.67 | 43.64 | 45.01 | 47.20 | 42.94 | 37.66 | 43.87 |
| | 0.03 | 69.37 | 60.02 | 55.76 | 43.95 | 46.07 | 44.77 | 46.88 | 43.79 | 39.77 | 34.21 |
| | 0.02 | 67.38 | 55.90 | 52.96 | 45.93 | 47.04 | 29.34 | 38.80 | 30.67 | 34.30 | 27.47 |
| | 0.01 | 61.94 | 57.29 | 55.60 | 43.23 | 42.45 | 37.81 | 29.81 | 32.02 | 28.98 | 38.13 |
| | 0 | 57.95 | 49.45 | 36.59 | 37.03 | 31.31 | 13.55 | 28.18 | 24.66 | 15.81 | 11.38 |
| | 0 | 51.68 | 46.86 | 37.27 | 27.77 | 17.57 | 10.18 | 1.52 | −2.42 | −0.69 | 0 |

### OTHER EMBODIMENTS

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in cellular and molecular biology, pharmacology, immunology, or related fields are intended to be within the scope of the invention.

What is claimed is:

1. A method for treating a patient diagnosed with or at risk for developing an inflammatory skin disorder, said method comprising administering to said patient a prostaglandin and a steroid, wherein said prostaglandin and said steroid are topically administered simultaneously or within fourteen days of each other, in amounts sufficient to treat said patient.

2. The method of claim 1, wherein said prostaglandin is alprostidil, misoprostil, dinoprostone, prostaglandin E2, prostaglandin A1, prostaglandin A2, prostaglandin B1, prostaglandin B2, prostaglandin D2, prostaglandin F1α, prostaglandin F2α, prostaglandin I1, prostaglandin-ici 74205, prostaglandin F2β, 6-keto-prostaglandin F1α, prostaglandin E1 ethyl ester, prostaglandin E1 methyl ester, prostaglandin F2 methyl ester, arbaprostil, omoprostil, 13,14-dihydroprostaglandin F2α, or prostaglandin J.

3. The method of claim 2, wherein said prostaglandin is alprostidil.

4. The method of claim 1, wherein said prostaglandin is alprostidil and said steroid is diflorasone.

5. A method for treating a patient diagnosed with or at risk for developing an inflammatory skin disorder, said method comprising administering to said patient a beta-adrenergic receptor ligand and a steroid, wherein said beta-adrenergic receptor ligand and said steroid are topically administered simultaneously or within fourteen days of each other, in amounts sufficient to treat said patient.

6. The method of claim 5, wherein said beta-adrenergic receptor ligand is isoproterenol, dobutamine, metaproterenol, terbutaline, isoetharine, finoterol, formoterol, procaterol, ritodrine, salmeterol, bitolterol, pirbuterol, albuterol, levalbuterol, epinephrine, ephedrine, propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, or propafenone.

7. The method of claim 6, wherein said beta-adrenergic receptor ligand is isoproterenol.

8. The method of claim 5, wherein said beta-adrenergic receptor ligand is isoproterenol and said steroid is prednisolone.

9. A method for treating a patient diagnosed with or at risk for developing an inflammatory skin disorder, said method comprising administering to said patient an anti-mitotic agent and a steroid, wherein said anti-mitotic agent and said steroid are topically administered simultaneously or within fourteen days of each other, in amounts sufficient to treat said patient.

10. The method of claim 9, wherein said anti-mitotic agent is podofilox, etoposide, teniposide, or griseofulvin.

11. The method of claim 10, wherein said antimitotic agent is podofilox.

12. The method of claim 9, wherein said anti-mitotic agent is podofilox and said steroid is dexamethasone.

13. A method for treating a patient diagnosed with or at risk for developing an inflammatory skin disorder, said method comprising administering to said patient a microtubule inhibitor and a steroid, wherein said microtubule inhibitor and said steroid are topically administered simultaneously or within fourteen days of each other, in amounts sufficient to treat said patient.

14. The method of claim 13, wherein said microtubule inhibitor is an alkaloid, paclitaxel, or docetaxel.

15. The method of claim 14, wherein said alkaloid is colchicine or a vinca alkaloid.

16. The method of claim 15, wherein said vinca alkaloid is vinblastine, vincristine, vinorelbine, or vindesine.

17. The method of claim 13, wherein said microtubule inhibitor is colchicine and said steroid is dexamethasone.

18. The method of claim 1, wherein said steroid is dexamethasone, diflorasone, flumethasone, or prednisolone.

19-26. (canceled)

27. The method of claim 1, wherein said combination is administered within ten days of each other.

28. The method of claim 27, wherein said combination is administered within five days of each other.

29. The method of claim 28, wherein said combination is administered within twenty-four hours of each other.

30. The method of claims 1, wherein said inflammatory skin disorder is psoriasis, inflammatory dermatosis, or atopic dermatosis.

31. A pharmaceutical composition comprising a prostaglandin, a steroid, and a pharmaceutically acceptable carrier, wherein said prostaglandin and said steroid are present in amounts that, when topically administered to a patient, reduce or prevent the symptoms of an inflammatory skin disorder.

32-40. (canceled)

41. A method of producing a pharmaceutical composition for treating an inflammatory skin disorder, said method comprising admixing a prostaglandin, a steroid, and a pharmaceutically acceptable carrier, wherein said prostaglandin and said steroid are present in amounts that, when topically administered to a patient, reduce or prevent the symptoms of an inflammatory skin disorder.

42-81. (canceled)
